(19) United States
(12) Patent Application Publication
(10) Pub. No.: US 2016/0199308 A1
(43) Pub. Date: Jul. 14, 2016

(54) MAGNETIC NANO CORE-SHELL CAPSULE AND APPLICATION THEREOF

(71) Applicant: National Tsing Hua University, Hsinchu (TW)

(72) Inventors: Shang-Hsiu Hu, Hsinchu (TW); Yun-Ting Li, Hsinchu (TW); Jen-Hung Fang, Hsinchu (TW)

(21) Appl. No.: 14/661,592

(22) Filed: Mar. 18, 2015

(30) Foreign Application Priority Data
Jan. 13, 2015 (TW) 104101090

(51) Int. Cl. A61K 9/48; A61K 31/704; A61K 31/337; A61K 49/18

(52) U.S. Cl. CPC A61K 9/485; A61K 49/1827; A61K 31/704; A61K 31/337; A61K 9/4866

(57) ABSTRACT

The present invention provides a magnetic nano core-shell capsule for drug delivery, which including: a plurality of amphiphilic protein, a plurality of iron oxide nanoparticles, a hydrophilic drug and a hydrophobic drug. Wherein the magnetic nano core-shell capsule has good biocompatibility duo to the amphiphilic protein as the material, and it only needs single step emulsion to form its nano hollow structure. Therefore, the magnetic nano core-shell capsule of the present invention has high drug loading capacity, the ability of encapsulating hydrophobic and hydrophilic pharmaceuticals simultaneously and characteristic of controlled-release drug delivery. Thus it can be used for targeted drug delivery, magnetic resonance imaging and hyperthermia.

### CROSS-REFERENCE TO RELATED APPLICATIONS

This application claims the priority of Taiwanese patent application No. 104101090 filed on 13 Jan. 2015, which is incorporated herewith by reference.

### BACKGROUND OF THE INVENTION

1. Field of the Invention

The invention relates to a magnetic nano core-shell capsule, more particularly, relates to a magnetic nano core-shell capsule for drug delivery.

2. The Prior Arts

The drug carrier is to delivery drug to the lesion in the human body. To assure the safety and efficacy of drug, the drug carrier system is widely used to improve drug release, absorption, distribution and metabolism. In recent years, protein and gene drug are widely used with the biotechnology development, but the protein drug delivery is easily damaged by stomach acid resulting in poor drug efficacy. On the other hand, the anticancer drug delivery can also damage the normal cells and cause more severe side effects because of the high toxicity thereof. Furthermore, the treatment of chronic illnesses commonly includes the long-term use of pharmacotherapy, a long-acting medication can reduce the frequency of medication-taking, which are the key point considerations for the drug carrier system development.

In the drug carrier technology, nano-delivery system is widely used in pharmaceutical and biotechnology industries. The core-shell nanostructure formed by the organic material is used to be a drug carrier, such as liposome formed by double layers of lipid or micelle formed by amphoteric polymers, however, the organic core-shell nanostructure has some problem with instability, uncontrollability and complex forming process. Additionally, these technologies use polymers as material and several small molecular surface active agents to stabilize the interface of the core-shell nanostructure, which are extremely toxic for human body.

The current anticancer drugs have some clinical limitations due to toxicity to normal cells. Therefore, it needs to develop a drug carrier with low-invasive treatment for easy to use, which can increase patient convenience to take, and improve the availability of drug in the body to get best outcome, while the drug carrier also can reduce side effects to improve the discomfort caused by drug.

### SUMMARY OF THE INVENTION

Accordingly, the present invention provides a magnetic nano core-shell capsule, which has a hollow nanostructure formed by one-step emulsification of amphiphilic proteins and iron oxide (Fe₃O₄) nanoparticles. The amphiphilic protein can be a stabilizer and a surface active agent to form a drug carrier with high encapsulation efficiency. Preferably, the magnetic nano core-shell capsule has the ability of encapsulating hydrophobic and hydrophilic pharmaceuticals simultaneously. The synthesis of the magnetic nano core-shell capsule is to use the one-step emulsification which simplifies many emulsification steps and the use of surface active agents to stabilize the interface in the conventional techniques. The magnetic nano core-shell capsule of the present invention only needs the formation of one-step emulsification of amphiphilic protein and iron oxide (Fe₃O₄) nanoparticles; it has an excellent biocompatibility and multifunctionality for drug delivery.

An object of this invention is to provide a magnetic nano core-shell capsule for drug delivery, comprising: a plurality of amphiliphilic proteins, wherein the amphiliphilic protein has a hydrophilic end and a hydrophobic end; a plurality of iron oxide (Fe₃O₄) nanoparticles; a hydrophilic drug; and a hydrophobic drug, wherein the magnetic nano core-shell capsule has an aqueous phase core layer, encapsulating the hydrophilic drug; and an oil phase shell layer, encapsulating the plurality of iron oxide nanoparticles and the hydrophobic drug, and the amphiliphilic proteins are between the aqueous phase core layer and the oil phase shell layer to surround the oil phase shell layer; wherein the ratio of the amphiliphilic proteins and the iron oxide nanoparticles is 1:0.8 to 1:16.7.

In one embodiment, the hydrophilic ends of the amphiliphilic proteins form the aqueous phase core layer, and the hydrophobic ends and the hydrophilic ends of the amphiliphilic proteins surround to form the oil phase shell layer

In one embodiment, the hydrophobic ends of the amphiliphilic proteins and iron oxide nanoparticles are tight-knit in the oil phase shell layer.

In one embodiment, the amphiliphilic protein is bovine serum albumin or lactoferrin.

In one embodiment, the iron oxide nanoparticles provide the magnetic nano core-shell capsule the ability of magnetic manipulation.

In one embodiment, the magnetic nano core-shell capsule is applied in a drug delivery system.

In one embodiment, the magnetic nano core-shell capsule is applied in a nuclear magnetic resonance imaging agent.

Another object of this invention is to provide a method of manufacturing a magnetic nano core-shell capsule, comprising the steps of: mixing a plurality of amphiliphilic proteins with a hydrophilic drug to form a solution of aqueous phase core layer; dispersing a plurality of iron oxide nanoparticles in CHCl₃ solution to form a solution of oil phase shell layer; emulsifying the solution of aqueous phase core layer and the solution of oil phase shell layer to form a emulsion solution; and removing the CHCl₃ solution from the emulsion solution to obtain the magnetic nano core-shell capsule, wherein the emulsifying step is performed one time.

Another object of this invention is to provide a magnetic nano core-shell capsule for drug delivery, comprising: a plurality of amphiliphilic proteins, wherein the amphiliphilic protein has a hydrophilic end and a hydrophobic end; a plurality of iron oxide (Fe₃O₄) nanoparticles; a hydrophilic drug; and a hydrophobic drug, wherein the hydrophilic ends of the amphiliphilic proteins form a first space, the hydrophobic ends of the amphiliphilic proteins surround the hydrophobic end of the amphiliphilic proteins to form a second space, the second space surrounds the first space, and the hydrophilic drug is in the first space, and iron oxide nanoparticles and the hydrophobic drug are in the second space; wherein the first space forms an aqueous phase core layer, the second space forms an oil phase shell layer, and the hydrophilic drug is in the aqueous phase core layer, the hydrophobic drug is in the oil phase shell layer; and wherein the ratio of the amphiliphilic proteins and the iron oxide nanoparticles is 1:0.8 to 1:16.7.

The magnetic nano core-shell capsule of the present invention with hollow nanostructure contains an aqueous phase core layer and an oil phase shell layer; so it has the ability of encapsulating hydrophobic and hydrophilic pharmaceuticals simultaneously. The hydrophobic end of the amphiliphilic protein and iron oxide nanoparticles are tight-knit in the hydrophobic oil phase shell layer, iron oxide nanoparticles are used to stabilizer the oil phase shell layer in the emulsification step. Due to iron oxide nanoparticles-containing, the oil phase shell layer of the magnetic nano core-shell capsule can increase structural integrity to protect the drug and have the ability of magnetic manipulation to be used in hyperthermia and drug release carrier under magnetic field treatment.

The protein in the magnetic nano core-shell capsule is denatured after heating to lead to aggregate effect, and to have high degree of drug accumulation and better targeted therapy. In addition, the magnetic nano core-shell capsule can be re-dispersed after repeatedly centrifuge and wash to exhibit the excellent stability. The hydrophobic oil phase shell layer is formed the amphiliphilic protein and iron oxide nanoparticles tight-knit, so the layer not only stabilizes the structure, but also creates a highly hydrophobic environment to prevent the hydrophilic drug in the aqueous phase core layer release, and to carry the hydrophobic drug as well. Moreover, the phase shell layer can increase the ability to target drug delivery and change the property of the drug carrier by modifying the surface of the oil phase shell layer to improve the cellular uptake efficiency or the accuracy of lesion. In the present invention, the magnetic nano core-shell capsule is of considerable help for drug treatment.

The detailed technology and above preferred embodiments implemented for the present invention are described in the following paragraphs accompanying the appended drawings for people skilled in this field to well appreciate the features of the claimed invention.

### BRIEF DESCRIPTION OF THE DRAWINGS

FIG. 1 is a view shows the magnetic nano core-shell capsule of the present invention.

FIG. 2 shows Bovine serum albumin (BSA) having the hydrophobic domain to carry fatty acid, thyroxine, diazepam and calcium.

FIG. 3 is scanning electron microscopy (SEM) image of the magnetic nano core-shell capsule according to the first embodiment of the present invention.

FIGS. 4 (a) to (d) are a transmission electron microscope (TEM) images of the magnetic nano core-shell capsule according to the first embodiment of the present invention, which are taken after washing with deionized water and drying under a vacuum, (a) and (b) show that the fold portion of the oil phase core-shell is darker, (c) and (d) show the iron oxide tight-knit with BSA to form a stable oil phase core-shell.

FIG. 5 is scanning electron microscopy (SEM) image of the magnetic nano core-shell capsule according to the second embodiment of the present invention.

FIGS. 6 (a) to (d) are a transmission electron microscope (TEM) images of the magnetic nano core-shell capsule according to the second embodiment of the present invention, which are taken after washing with deionized water and drying under a vacuum, (a) and (b) show that the fold portion of the oil phase core-shell is darker, (c) and (d) show that the concentration of lactoferrin can be modified to form a solid spherical structure.

FIGS. 7 (a) to (d) are graphs to compare the hollow spherical structure (VN) with the solid spherical structure (SEP) of the magnetic nano core-shell capsule; (a) size distribution of the resulting VN and SEP; (b) the loading capacity, the hydrophilic doxorubicin (Doxo) and the hydrophobic paclitaxel (PTX) are encapsulated into the VN and SEP, (c) the cumulative release from VNs-Doxo/PTX and SEP-Doxo-PTX, (d) cellular uptake of the VN in the Hela cells, quantum dots (QDs) show with white arrows in the cytoplasm after a 2h incubation.

FIGS. 8 (a) to (c) are scanning electron microscopy (SEM) images of the magnetic nano core-shell capsule according to different concentration of bovine serum albumin (BSA).

FIGS. 9 (a) to (e) show that the concentration of BSA and Fe₃O₄ can be adjusted to change the diameter of the magnetic nano core-shell capsule; (a) and (b) show the structure of the magnetic nano core-shell capsule formed at the condition of 1 wt % BSA and 8.35% wt % Fe₃O₄ by dynamic light scattering (DLS); (c) and (d) show the structure of the magnetic nano core-shell capsule formed at the condition of 2 wt % BSA and 16.7 wt % Fe₃O₄ by DLS; (e) shows the size distribution of the magnetic nano core-shell capsule.

FIG. 10 shows the size distribution of the magnetic nano core-shell capsule with different amount of Fe₃O₄ containing.

FIGS. 11 (a) to (f) show the morphology, surface potential and hydrodynamic radius of the magnetic nano core-shell capsule at different temperature; (a) to (d) are the aggregate effect images of the magnetic nano core-shell capsule at high temperature by scanning electron microscopy; (a) and (c) are at room temperature; (b) and (d) heat to 60° C.; (e) shows the surface potential of the magnetic nano core-shell capsule at different temperature; (f) shows the size distribution of the magnetic nano core-shell capsule at different pH value.

FIG. 12 shows hydrated radius of the magnetic nano core-shell capsule at different pH value.

FIG. 13 shows the survival rate of lung cancer A549 cell line supplemented with different concentration of the magnetic nano core-shell capsule; the control group is without supplementing any magnetic nano core-shell capsule.

FIG. 14 shows that cervical cancer HeLa cell line is cultured with 10 mg/mL high and 3 mg/mL low concentration of the magnetic nano core-shell capsule; quantum dots (QDs) show with white arrows.

FIGS. 15 (a) and (b) show the sensitive magnetic properties of the magnetic nano core-shell capsule; (a) show the release rate of the magnetic nano core-shell capsule under the exposure of a high-frequency magnetic field (MF) (50 kHz) at 8 and 16 kA m⁻¹; (b) show in the release rate of the magnetic nano core-shell capsule is controllable in the on-off magnetic field. 16 ka/m-Doxo and 8 ka/m-Doxo are the magnetic nano core-shell capsule encapsulating the hydrophilic drug (Doxo) under MF treatment at 16 kA m⁻¹ or 8 kA m⁻¹ for 10 min, 16 ka/m-PTX and 8 ka/m-PTX are the magnetic nano core-shell capsule encapsulating the hydrophobic drug (PTX) under MF treatment at 16 kA m⁻¹ or 8 kA m⁻¹ for 10 min; DE-Doxo and DE-PTX are the magnetic nano core-shell capsule encapsulating Doxo or PTX without MF treatment.

FIGS. 16 (a) and (b) show in vivo targeting ability of the magnetic nano core-shell capsule; (a) shows that the magnetic nano core-shell capsule accumulates in tumor site; (b) shows that the magnetic nano core-shell capsule under MF treatment for 30 day to inhibit the tumor growth in mouse. PD-VNs is the magnetic nano core-shell capsule encapsulating the hydrophilic drug (Doxo) and hydrophobic drug (PTX); PD-VNs-MFx1 and PD-VNs-MFx3 are the magnetic nano core-shell capsule encapsulating hydrophobic drug (PTX) and the hydrophilic drug (Doxo) under MF treatment once and three times, respectively; PD is free hydrophilic drug (Doxo) and hydrophobic drug (PTX); VNs+MF is the magnetic nano core-shell capsule under MF treatment; PD-VNs is the magnetic nano core-shell capsule encapsulating hydrophobic drug (PTX) and the hydrophilic drug (Doxo); control is physiological saline solution.

### DETAILED DESCRIPTION OF THE PREFERRED EMBODIMENT

In the present invention, the magnetic nano core-shell capsule is synthesized by single amphiphilic protein and iron oxide (Fe₃O₄) nanoparticles via on-step emulsification to form nano-hollow structure. The drug can be directly encapsulated in the synthesis of the magnetic nano core-shell capsule to increase the loading capacity. The magnetic nano core-shell capsule has the abilities of encapsulating hydrophobic and hydrophilic pharmaceuticals simultaneously to control drug release; it also can be applied in magnetic resonance imaging and hyperthermia. And the magnetic nano core-shell capsule can be simply modified to have the ability to target drug delivery.

### Example 1

### Synthesis of the Magnetic Nano Core-Shell Capsule

After the consideration of the biological use of volatile solvents, the magnetic nano core-shell capsule of the present invention is synthesized by single amphiphilic protein and iron oxide (Fe₃O₄) nanoparticles via simple on-step emulsification to form nano-hollow structure. Comparing with conventional techniques of forming a hollow structure, it needs many emulsification steps, which comprising: first emulsification is to form water-in-oil (W/O) structure, and second emulsification is to from water-in-oil-in water (W/O/W). However, in the present invention, the shell layer and the core layer of the magnetic nano core-shell capsule are all synthesized by the amphiphilic protein; it only needs one-step emulsification to form W/O/W hollow structure. The amphiphilic protein combines with the iron oxide nanoparticles to stabilize an oil phase shell layer, have the ability of encapsulating hydrophobic and hydrophilic pharmaceuticals simultaneously.

### 1.1 Bovine Serum Albumin (BSA) as an Example of Single Amphiphilic Protein

In this embodiment, bovine serum albumin and oil nano iron oxide (preferably nano magnetic iron), the hydrophilic drug doxorubicin hydrochloride (DOXO) and the hydrophobic drug paclitaxel (PTX) as an example of material, describe the synthesis and application of the magnetic nano core-shell capsule of the present invention.

The synthesis of the magnetic nano core-shell capsule is as follows:
1. Prepare BSA solution, DOXO solution and PTX solution.
2. Synthesize 5 nm oil iron oxide (Fe₃O₄), remove the ethanol from the Fe₃O₄, and dispersing Fe₃O₄ in CHCl₃ solution, then add PTX solution to obtain an oil phase material.
3. Use BSA solution and DOXO solution as a water phase material mixing with the oil phase material to form a mixture, and the mixture is emulsified by ultrasonication for 30 sec. After completely emulsification, stir the mixture at room temperature to evaporate CHCl₃ solution in ventilation station, and then obtain a product mixture.
4. Wash the product mixture by Milli-Q water, collect the magnetic nano core-shell capsule after centrifugation at 8,000 rpm and repeat 3 times, then wash the magnetic nano core-shell capsule by Milli-Q water.

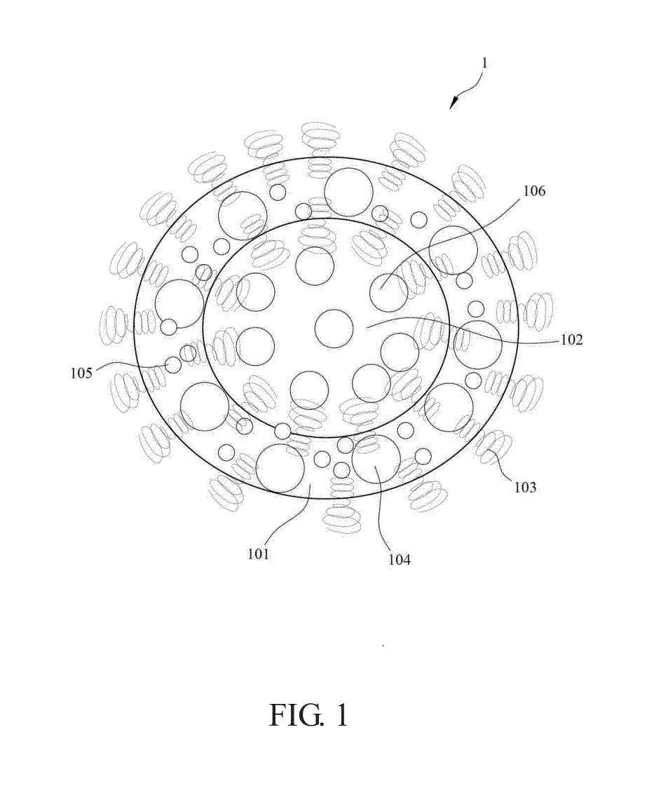

Through above mentioned steps, the magnetic nano core-shell capsule 1 of the present invention is formed as a hollow structure, as shown in FIG. 1, the structure was the hydrophilic end of amphiphilic protein 103, preferably BSA, surrounding to form an aqueous phase core layer 102, and an oil phase shell layer 103 surrounding the aqueous phase core layer 102. Wherein the oil phase shell layer 101 was formed by the amphiliphilic protein 103, preferably BSA, tight-knit with iron oxide nanoparticles 104, so the oil phase shell layer 103 can stabilize the core-shell structure of the magnetic nano core-shell capsule 1, and also create a highly hydrophobic environment of oil phase shell layer to prevent the hydrophilic drug 106 in the aqueous phase core layer 102 release, and to carry the hydrophobic drug 105 as well. The iron oxide nanoparticles 104 give the magnetic nano core-shell capsule 1 the ability of magnetic manipulation.

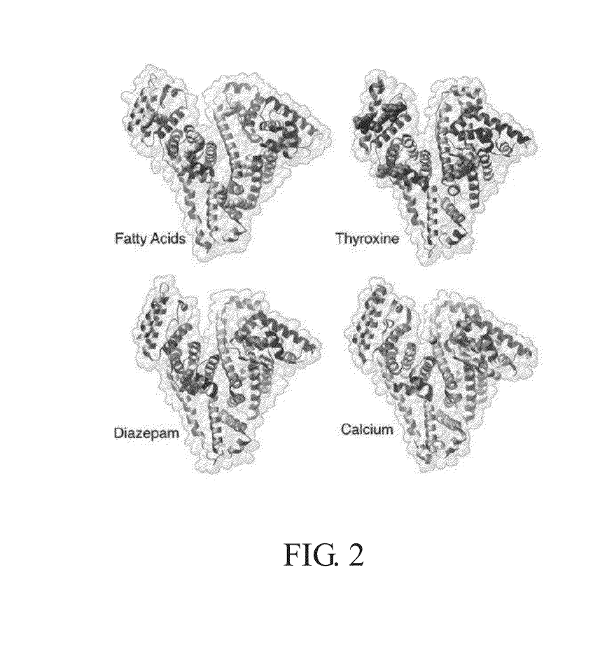

The sequence of the amphiliphilic protein such as BSA demonstrates the hydrophilic and the hydrophobic domain to carry an oil phase molecule, such as fatty acid, thyroxine, diazepam and calcium, as shown in FIG. 2, the oil phase iron oxide can bind to these sites; many hydroxyl functional groups of amino acids in the hydrophobic ends are toward aqueous solution, BSA is between two phase. According to hydrophilic-lipophilic balance value (HLB), BSA has the ability to stable oil/water (HLB value: 8-16) and water/oil (HLB value: 3-8) at a specific concentration; it can be used as water/oil and oil/water emulsifier, therefore, self-assembly form water-oil-water hollow structure.

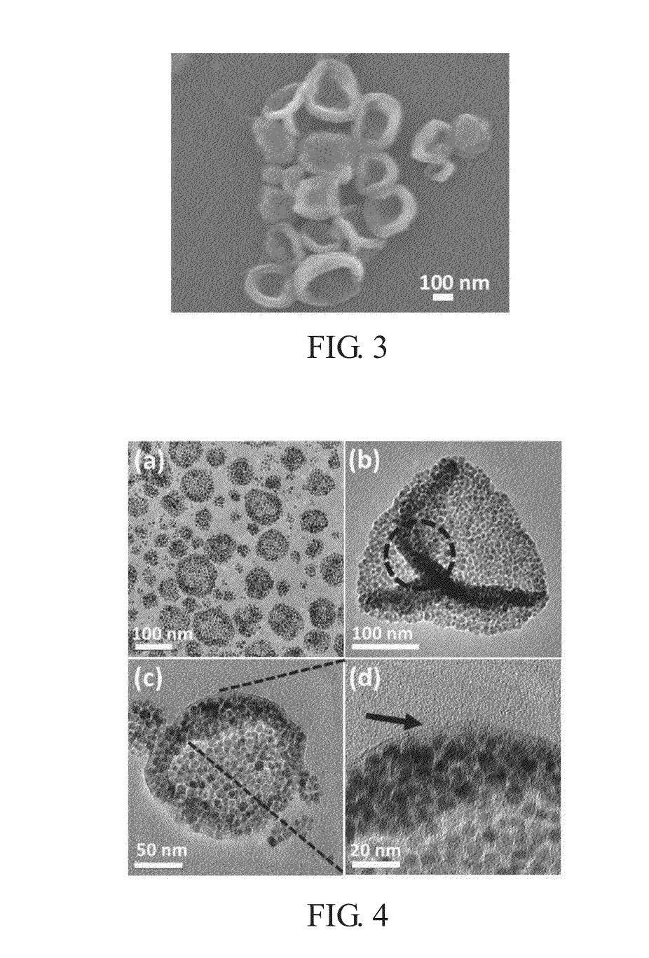

The structure of the magnetic nano core-shell capsule of the present invention are examined by scanning electron microscopy (SEM) and transmission electron microscope (TEM), as shown in FIGS. 3 and 4. FIG. 3 is SEM image of the magnetic nano core-shell capsule of the present invention, which is taken after washing with deionized water and drying under a vacuum. The morphology of dried magnetic nano core-shell capsule is obvious collapse or shrinkage because protein cannot have a structural rigidity and iron oxide in oil phase cannot evaporate, which all demonstrate that the structure inside the magnetic nano core-shell capsule is an aqueous phase indeed. FIGS. 4(a) to (d) are TEM images of the magnetic nano core-shell capsule of the present invention, which can clearly observe the perspective structure of the magnetic nano core-shell capsule after drying shrinkage. The darker black particles in the image are iron oxide, and a relatively light part is BSA in a layer outside the iron oxide (FIGS. 4 (a) and (b)), the iron oxide is tight-knit with BSA to form a flexible oil phase core-shell (FIGS. 4 (c) and (d)).

### 1.2 Lactoferrin (Lf) as an Example of Single Amphiphilic Protein

In this embodiment, lactoferrin and oil nano iron oxide (preferably nano magnetic iron), the hydrophilic drug doxorubicin hydrochloride (DOXO) and the hydrophobic drug paclitaxel (PTX) as an example of material, describe the synthesis and application of the magnetic nano core-shell capsule of the present invention, the method of synthesizing as the description in EXAMPLE 1.1.

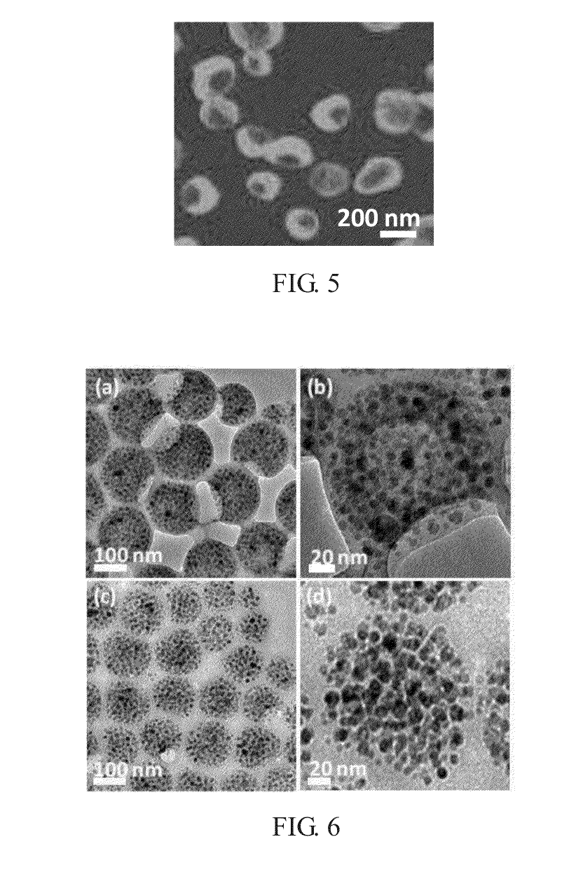

Through above mentioned steps, the magnetic nano core-shell capsule of the present invention is formed as a hollow and core-shell structure, which is a complex formed by lactoferrin and iron oxide. As shown in FIG. 5, the nano-structure of the magnetic nano core-shell capsule remains original properties after encapsulating the hydrophilic doxorubicin and hydrophobic paclitaxel, and the hollow structure is collapse, the results all show the same with EXAMPLE 1.1.

However, the diameter of the magnetic nano core-shell capsule synthesized by lactoferrin is different from BSA, to observe under transmission electron microscope, as shown in FIGS. 6 (a) to (d). The diameter of the magnetic nano core-shell capsule synthesized by lactoferrin is about 160 nm, and the fold portion of the oil phase core-shell is darker in the image after drying under a vacuum (FIGS. 6 (a) to (b)). In the present invention, the proportional of lactoferrin can be modified, for example, the concentration of lactoferrin can be decreased to form a solid spherical structure (FIGS. 6 (c) to (d)).

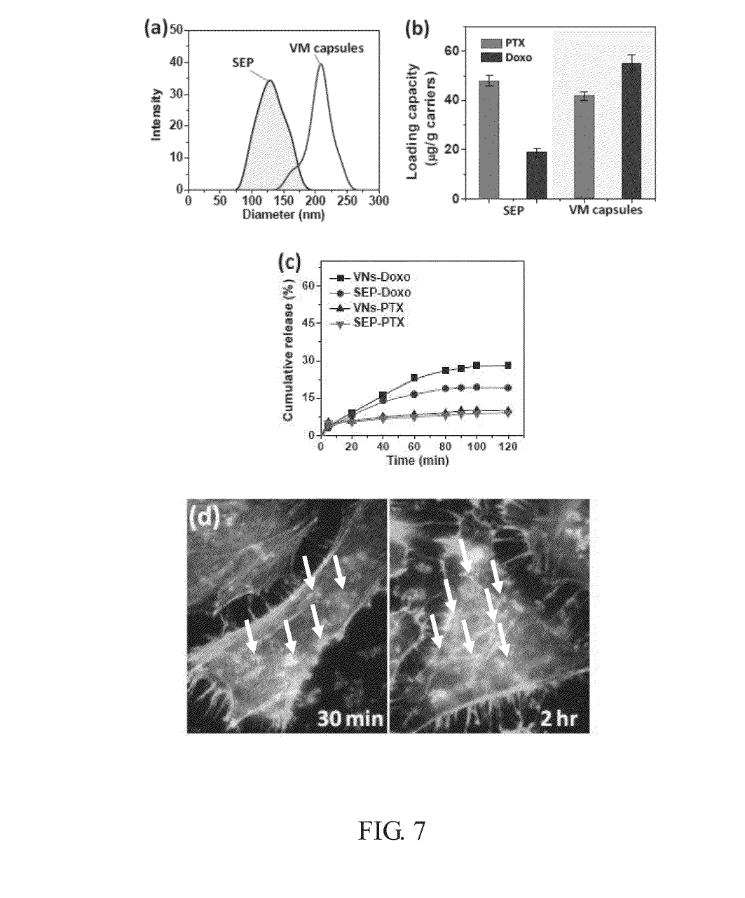

The diameter of the magnetic nano core-shell capsule of the present invention is determined to comparing the hollow spherical structure (VN) with the solid spherical structure (SEP) by modifying the proportion of the lactoferrin, 0.5 wt %-8 wt % lactoferrin mixing with iron oxide forms VN, and 0.1 wt %-0.5 wt % lactoferrin mixing with iron oxide forms SEP. TEM analysis demonstrates that the size of the VNs is significantly larger than that of the SEP. This result was additionally investigated by dynamic light scattering (DLS). The particle sizes are shown in FIG. 7 (a) VNs are larger than the SEP, and the VNs and the SEP have a mean diameter of about 220 nm and 130 nm, respectively. On closer observation, the VNs have a mixed morphology, which contains their two size distributions. Additionally, the sizes of the emulsion particles before drying estimated using DLS are slightly larger than these of the dried particles observed using TEM. This indicates the relatively lower shrinkage during drying, and thus the rigidity of the iron oxide-reinforced Lf. Next, the hydrophilic Doxo and the hydrophobic PTX are encapsulated into the VNs and the SEP, separately (forming VNs-PTX, VNs-Doxo, VNs-PTX/Doxo, SEP-PTX, SEP-Doxo, SEP-PTX/Doxo). In FIG. 7 (b), the loading capacity of PTX is similar for both VNs (42 μg g⁻¹ carrier) and SEPs (48 ng g⁻¹ carrier). Because PTX, which has a low water solubility, is dissolved in the organic phase during emulsification, it prefers to reside in the hydrophobic domain of the VNs and SEPs. In contrast, the loading capacity of Doxo is higher for the VNs (55 μg g⁻¹ carrier) than for the SEPs (18 μg g⁻¹ carrier). For cumulative release, as shown in FIG. 7 (c), the two structures demonstrate a slow release profile that does not decrease the release rate until 1 h. In addition, to verify the cellular uptake of the VN capsules in HeLa cells, as shown in FIG. 7 (d), which displays that few VNs remain at the cell surfaces when incubates for 30 min with HeLa cells; however, the VNs are rapidly internalized and accumulated in the cytoplasm after 2 h, quantum dots (QDs) show with white arrows in the cytoplasm. The result validates that the magnetic nano core-shell capsule of the present invention can be applied in targeted therapy.

### 1.3 The Effect of the Concentration of the Amphiphilic Protein in the Magnetic Nano Core-Shell Capsule of the Present Invention

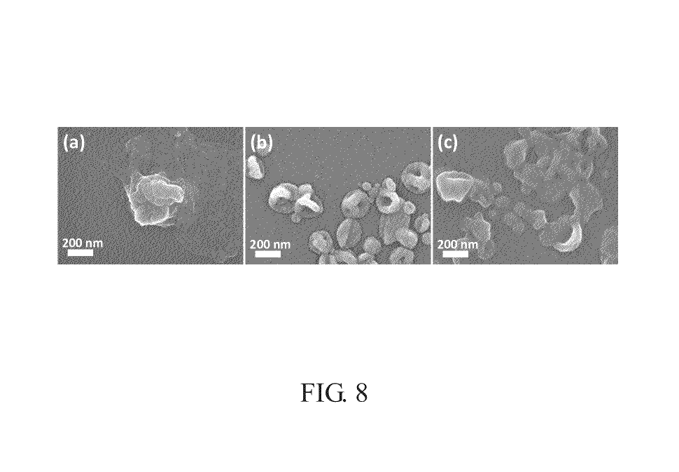

In above mentioned steps, to test whether the magnetic nano core-shell capsule can be formed, at the concentration of iron oxide is 0.017 g/mL, the weight percent concentration of amphiphilic protein is <1 wt %, 1 wt %-4 wt % and >4 wt %, respectively. The results showing bovine serum albumin (BSA) as amphiphilic protein are listed in Table 1 and FIGS. 8 (a) to (c), the results showing lactoferrn as amphiphilic protein are listed in Table 2, which all demonstrate that the hollow structure is formed at 1 wt %-4 wt % amphiphilic protein, and the diameter of the magnetic nano core-shell capsule of the present invention is less than 200 nm (FIG. 8 (b)); and the magnetic nano core-shell capsule will not be formed when the weight percent concentration of amphiphilic protein is less or more (FIGS. 8 (a) and (c)).

TABLE 1
The effect of the concentration of the BSA in the magnetic nano
core-shell capsule

| BSA (wt%)/Fe₃O₄ (g/ml) | Diameter | FIG. | Quality |
|---|---|---|---|
| <1%/0.017 | >1000 nm | FIG. 8 (a) | Bad |
| 1%~4%/0.017 | >200 nm | FIG. 8 (b) | Good |
| >4%/0.017 | >300 nm | FIG. 8 (c) | Poor |

TABLE 2
The effect of the concentration of the lactoferrn in the magnetic nano
core-shell capsule

| Lactoferrin (wt%)/Fe₃O₄ (g/ml) | Diameter | Quality |
|---|---|---|
| <1%/0.017 | >1000 nm | Bad |
| 1%~4%/0.017 | >200 nm | Good |
| >4%/0.017 | >300 nm | Poor |

### Example 2

### The Diameter of the Magnetic Nano Core-Shell Capsule

In the embodiment, for a wider range of applications, the diameter of the magnetic nano core-shell capsule can be changed under artificial control by adjusting the concentration of Fe₃O₄, ambient temperature and pH value.

2.1 Adjust the Concentration of BAS and Fe₃O₄

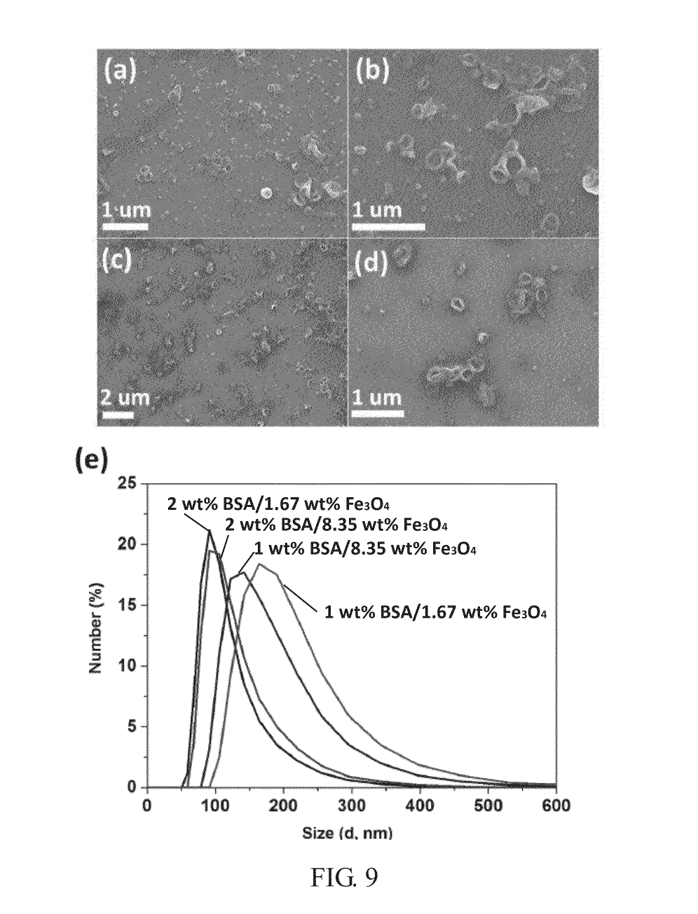

The concentration of BAS and Fe₃O₄ can be adjusted within the above-mentioned range to change the diameter of the magnetic nano core-shell capsule in EXAMPLE 1.1. In FIGS. 9 (a) and (d) show the structure of the magnetic nano core-shell capsule are investigated by dynamic light scattering (DLS); in FIGS. 9 (a) and (b) are the magnetic nano core-shell capsule formed at the condition of 1 wt % BSA and 8.35% wt % Fe₃O₄; FIGS. 9 (c) and (d) are the magnetic nano core-shell capsule formed at the condition of 2 wt % BSA and 16.7 wt % Fe₃O₄, which validate that the diameter of the magnetic nano core-shell capsule is increased with the increase of the concentration of Fe₃O₄. The size distribution of the magnetic nano core-shell capsule is shown in FIG. 9 (e).

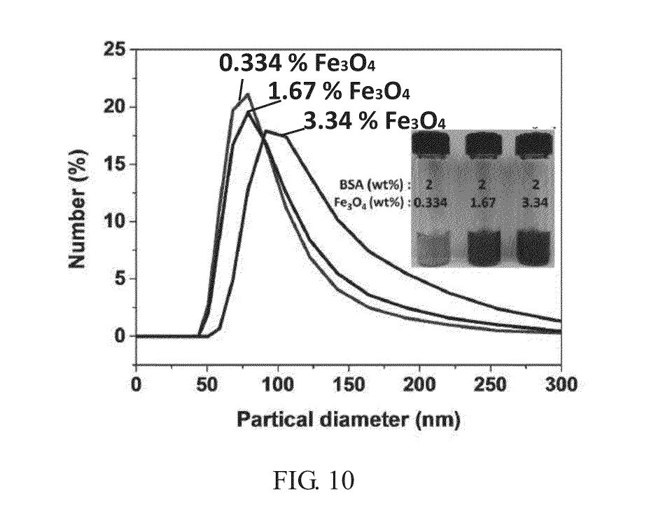

The magnetic nano core-shell capsule has different appearance with different amount of Fe₃O₄ containing. The diameter and size distribution of the magnetic nano core-shell capsule of the present invention can be changed by adjusting the concentration of Fe₃O₄, as shown in FIG. 10, the size distribution of the magnetic nano core-shell capsule is increased with the increase of the concentration of Fe₃O₄.

### 2.2 Adjust Temperature

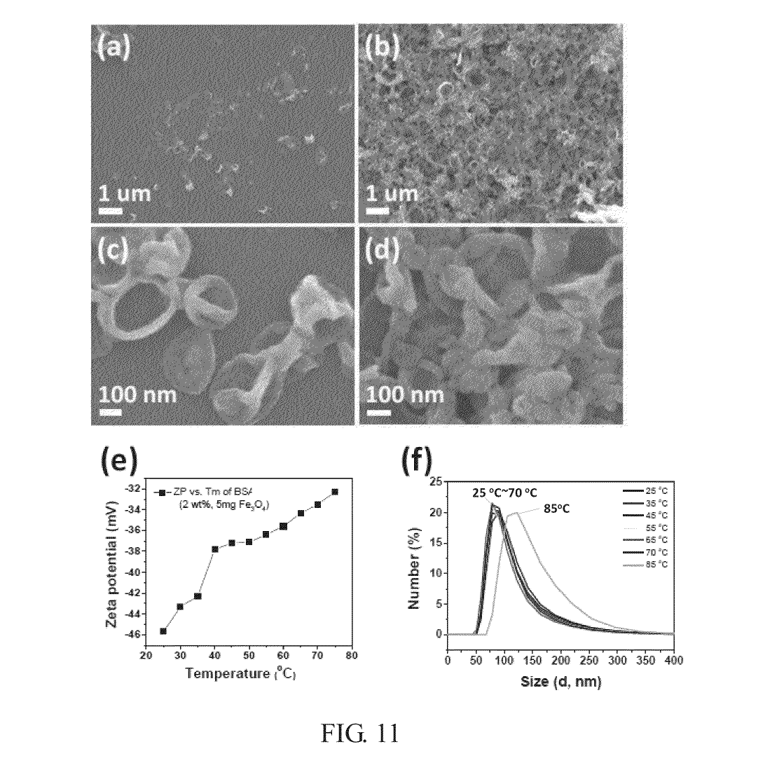

The morphology of the magnetic nano core-shell capsule of EXAMPLE 1.1 is different at different temperature, as shown FIGS. 11 (a) and (c), the magnetic nano core-shell capsule is dispersed at room temperature; as shown FIGS. 11 (b) and (d), but the magnetic nano core-shell capsule is aggregate when heating temperature up to 60° C. Thus, the magnetic nano core-shell capsule will be aggregate with higher temperature. Also, the electrical potential changes in the magnetic nano core-shell capsule at different temperature are determined by surface potential (zeta potential), as shown in FIG. 11(e), the magnetic nano core-shell capsule will be aggregate due to the electricity decreasing, when the ambient temperature of the magnetic nano core-shell capsule is increased. The hydrodynamic radius changes in the magnetic nano core-shell capsule at different temperature are measured by dynamic light scattering, the results validate that the protein in the magnetic nano core-shell capsule is denatured after heating to lead to aggregate effect, and to have high degree of drug accumulation and better targeted therapy.

### 2.3 Adjust pH Value

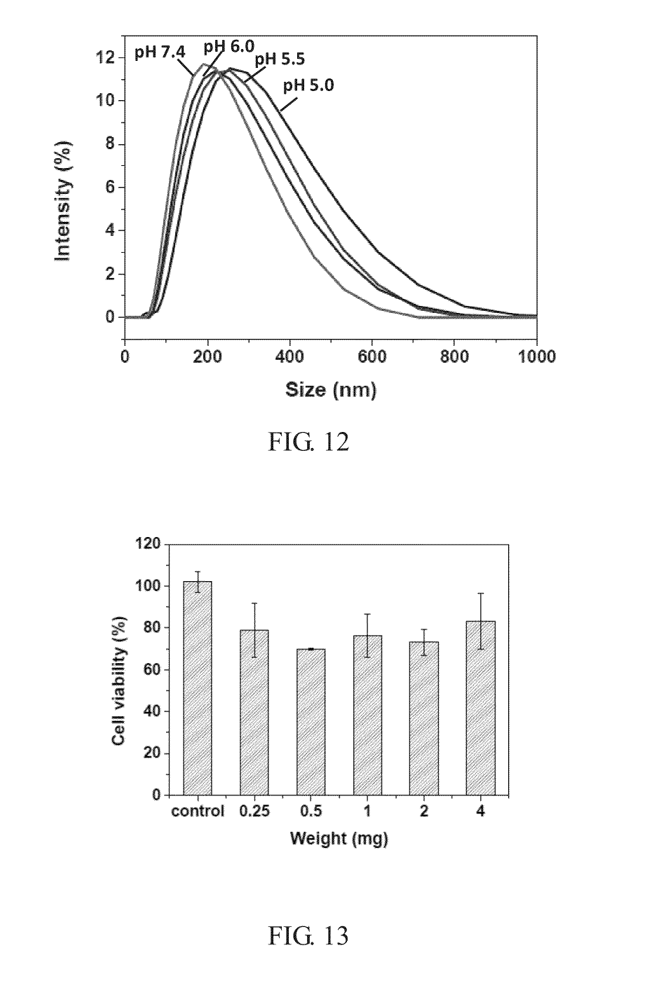

The hydrated radius of the magnetic nano core-shell capsule of EXAMPLE 1.1 is different at different pH value, as shown in Table 3 and FIG. 12, the hydrated radius is increased with pH value decreasing, and the hydrated radius can maintain the original structure of the magnetic nano core-shell capsule. The result validates that the structure of the magnetic nano core-shell capsule can maintain at an acidic ambient.

TABLE 3
The hydrated radius of the magnetic nano core-shell capsule at different
pH value

| | pH value | | | |
|---|---|---|---|---|
| | 7.4 | 6.0 | 5.5 | 5.0 |
| hydrated radius (nm) | 187.5 | 201.9 | 210.9 | 240.1 |

### Example 3

### Biocompatibility of the Magnetic Nano Core-Shell Capsule

The biocompatibility of the magnetic nano core-shell capsule of the EXAMPLE 1.1 is tested in the lung cancer A549 cell line. A549 cell line is cultured at different concentration of the magnetic nano core-shell capsule, as shown in FIG. 13, the survival rates of A549 cell line supplemented with the magnetic nano core-shell capsule groups are no difference from the control without supplemented any magnetic nano core-shell capsule. This result validates that the magnetic nano core-shell capsule has excellent biocompatibility.

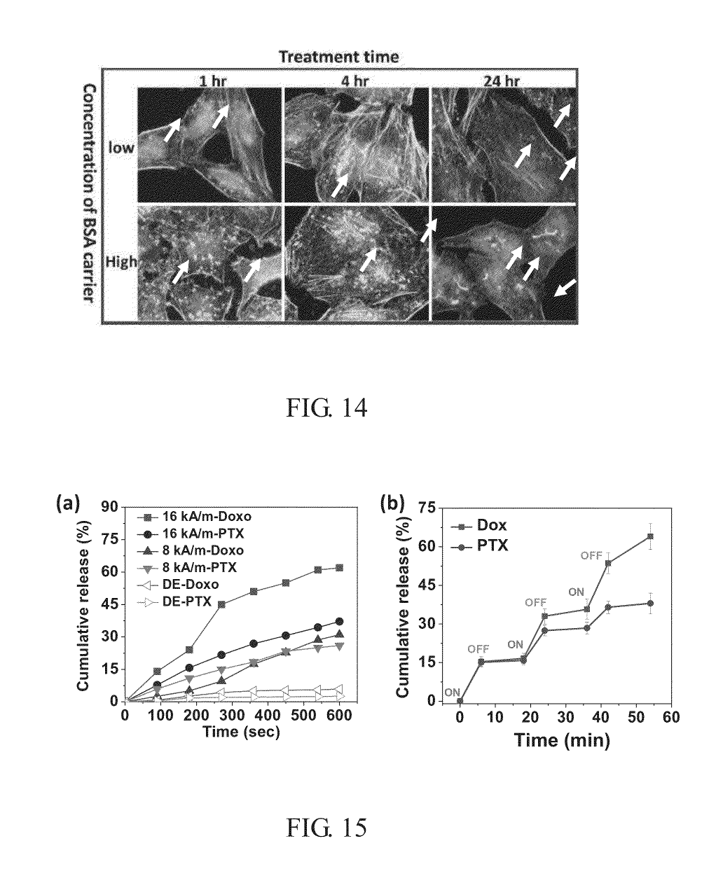

In addition, cervical cancer HeLa cell line is cultured with 10 mg/mL high and 3 mg/mL low concentration of the magnetic nano core-shell capsule of EXAMPLE 1.1. As shown in FIG. 14, the magnetic nano core-shell capsule quantum dots (QDs) shows with white arrows, the cells can intake the magnetic nano core-shell capsule without any modification through endosomal compartment. When the concentration of the magnetic nano core-shell capsule is higher or the culture time is longer, the cumulative amount of the magnetic nano core-shell capsule will be increased in the cells. Therefore, the magnetic nano core-shell capsule has excellent biocompatibility.

### Example 4

### The Sensitive Magnetic Properties of the Magnetic Nano Core-Shell Capsule

To investigate the sensitive magnetic properties of the magnetic nano core-shell capsule, the magnetic nano core-shell capsule is under the exposure of a high-frequency magnetic field (MF) (50 kHz) at 8 and 16 kA m⁻¹. As shown in FIG. 15 (a), 16 kA/m-Doxo is the magnetic nano core-shell capsule encapsulating the hydrophilic drug (Doxo) under MF treatment at 16 kA m⁻¹ for 10 min, 16 kA/m-PTX is the magnetic nano core-shell capsule encapsulating the hydrophobic drug (PTX) under MF treatment at 16 kA m⁻¹ for 10 min. The MF obviously increases the release rate for both hydrophobic drug (PTX) and the hydrophilic drug (Doxo). And DE-Doxo and DE-PTX are the magnetic nano core-shell capsule encapsulating Doxo or PTX without MF treatment, and the release rate is slower than MF treatment. FIG. 15 (b) shows that the magnetic nano core-shell capsule encapsulating hydrophobic drug (PTX) or the hydrophilic drug (Doxo) in the on-off magnetic field increases the release rate with on-demand magnetic field.

A shorter MF exposure time at 8 kA m⁻¹ treat the magnetic nano core-shell capsule, and release of Doxo and PTX follow a staircase-shaped pattern, suggesting that the thermally induced release is an on-demand and stoppable process. The magnetic nano core-shell capsule is regulated by the MF reveal instant on/off-acting release and MF-intensity-dosage dependence, achieving it controllable.

### Example 5

### The In Vivo Targeting Ability of the Magnetic Nano Core-Shell Capsule

To evaluate the in vivo targeting ability of the magnetic nano core-shell capsule in EXAMPLE 1.1, 100 μL solution containing 1% wt/wt the magnetic nano core-shell capsule encapsulating hydrophobic drug (PTX) or the hydrophilic drug (Doxo) labeled by Cy 5.5 injects via tail vein of nude mice bearing HeLa tumor cells, the MF is applied once (PD-VNs-MFx1) and three times (PD-VNs-MFx3) respectively. The distribution of the tumor and the magnetic nano core-shell capsule is investigated by non-invasive Caliper IVIS system to observe the change in the animal. The present invention also provides only the injection of a free hydrophilic drug (Doxo) and hydrophobic drug (PTX) (PD), the magnetic nano core-shell capsule encapsulating the hydrophilic drug (Doxo) and hydrophobic drug (PTX) (PD-VNs) as comparison groups, as well as the injection of physiological saline solution as control group. In the embodiment, the mouse tumor size and weight are observed and measured by a fixed member to record three times a week. For the elliptical shape of the tumor, the tumor volume is measured by a vernier and calculated according to the following formula=½ab² (a: tumor size, b: tumor short diameter) to calculate tumor volume.

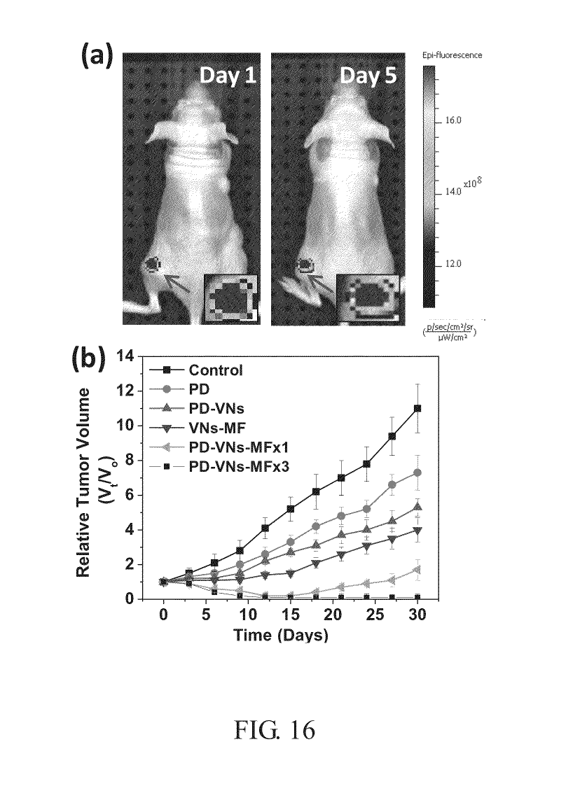

As shown in FIG. 16 (b), MF is applied once (PD-VNs-MFx1) and three times (PD-VNs-MFx3) for 30 day to inhibit the tumor growth, and PD-VNs-MFx3 has good treatment efficiency. The free hydrophilic drug (Doxo) and hydrophobic drug (PTX) (PD) do not have drug effect due to metabolic cause, and the tumor cells continue to proliferate. In addition, the magnetic nano core-shell capsule (drug-free) under MF treatment (VNs+MF) or the magnetic nano core-shell capsule encapsulating the hydrophilic drug (Doxo) and hydrophobic drug (PTX) (PD-VNs) without MF treatment show a little ability to inhibit the tumor growth. The control group has no inhibition for tumor cells due to the injection of physiological saline solution.

Moreover, the fluorescence dye is a model of the magnetic nano core-shell capsule encapsulating drug of the present invention to inject into mice, as shown in FIG. 16(a), the florescence image shows that the magnetic nano core-shell capsule accumulates in tumor site.

The results validate that the magnetic nano core-shell capsule of the present invention has good sensitive magnetic property for a drug control over a long period in vivo.

In summary, the magnetic nano core-shell capsule of the present invention is formed by general amphiphilic protein and iron oxide, wherein the amphiphilic protein has excellent biocompatibility and the iron oxide has the ability to control drug release, hyperthermia and nuclear magnetic resonance. Furthermore, the synthesis of the magnetic nano core-shell capsule is very simple by using a single amphiphilic protein to simultaneously stabilize the interfaces of oil/water and water/oil interface without complicated modification or polymerization, which is different from the traditional technical to increase the structure stability with the cross-linking reaction. Additionally, the step of encapsulating drug can also be completed in the synthesis process of the magnetic nano core-shell capsule; therefore, the magnetic nano core-shell capsule not only simplifies process, but also has a significantly higher encapsulation efficiency. The magnetic nano core-shell capsule have the ability to simultaneously encapsulate hydrophilic and hydrophobic drugs for a widely use.

What is claimed is:

1. A magnetic nano core-shell capsule for drug delivery, comprising:
a plurality of amphiliphilic proteins, wherein the amphiliphilic protein has a hydrophilic end and a hydrophobic end;
a plurality of iron oxide (Fe₃O₄) nanoparticles;
a hydrophilic drug; and
a hydrophobic drug,
wherein the magnetic nano core-shell capsule has an aqueous phase core layer, encapsulating the hydrophilic drug; and an oil phase shell layer, encapsulating the plurality of iron oxide nanoparticles and the hydrophobic drug, and the amphiliphilic proteins are between the aqueous phase core layer and the oil phase shell layer to surround the oil phase shell layer;
wherein the ratio of the amphiliphilic proteins and the iron oxide nanoparticles is 1:0.8 to 1:16.7.

2. The magnetic nano core-shell capsule according to claim 1, wherein the hydrophilic ends of the amphiliphilic proteins form the aqueous phase core layer, and the hydrophobic ends and the hydrophilic ends of the amphiliphilic proteins surround to form the oil phase shell layer.

3. The magnetic nano core-shell capsule according to claim 1, wherein the hydrophobic ends of the amphiliphilic proteins and the iron oxide nanoparticles are tight-knit in the oil phase shell layer.

4. The magnetic nano core-shell capsule according to claim 1, wherein the amphiliphilic protein is bovine serum albumin or lactoferrin.

5. The magnetic nano core-shell capsule according to claim 1, wherein the iron oxide nanoparticles provide the magnetic nano core-shell capsule the ability of magnetic manipulation.

6. The magnetic nano core-shell capsule according to claim 1 is applied in a drug delivery system.

7. The magnetic nano core-shell capsule according to claim 1 is applied in a nuclear magnetic resonance imaging agent.

8. A method of manufacturing a magnetic nano core-shell capsule according to claim 1, comprising the steps of:
mixing a plurality of amphiliphilic proteins with a hydrophilic drug to form a solution of aqueous phase core layer;
dispersing a plurality of iron oxide nanoparticles in CHCl₃ solution to form a solution of oil phase shell layer;
emulsifying the solution of aqueous phase core layer and the solution of oil phase shell layer to form an emulsion solution; and
removing the CHCl₃ solution from the emulsion solution to obtain the magnetic nano core-shell capsule,
wherein the emulsifying step is performed one time.

9. A magnetic nano core-shell capsule for drug delivery, comprising:
a plurality of amphiliphilic proteins, wherein the amphiliphilic protein has a hydrophilic end and a hydrophobic end;
a plurality of iron oxide (Fe₃O₄) nanoparticles;
a hydrophilic drug; and
a hydrophobic drug,
wherein the hydrophilic ends of the amphiliphilic proteins form a first space, the hydrophobic ends of the amphiliphilic proteins surround the hydrophobic end of the amphiliphilic proteins to form a second space, the second space surrounds the first space, and the hydrophilic drug is in the first space, and iron oxide nanoparticles and the hydrophobic drug are in the second space;
wherein the first space forms an aqueous phase core layer, the second space forms an oil phase shell layer, and the hydrophilic drug is in the aqueous phase core layer, the hydrophobic drug is in the oil phase shell layer; and
wherein the ratio of the amphiliphilic proteins and the iron oxide nanoparticles is 1:0.8 to 1:16.7.

10. The magnetic nano core-shell capsule according to claim 9, wherein the amphiliphilic protein is bovine serum albumin or lactoferrin.
